(19) United States
(12) Patent Application Publication
(10) Pub. No.: US 2014/0031881 A1
(43) Pub. Date: Jan. 30, 2014

(54) Ankle Nail Assembly

(71) Applicant: Osteomed LLC, Addison, TX (US)

(72) Inventor: Joel Bales, Addison, TX (US)

(73) Assignee: Osteomed LLC, Addison, TX (US)

(21) Appl. No.: 13/946,843

(22) Filed: Jul. 19, 2013

Related U.S. Application Data
(60) Provisional application No. 61/673,949, filed on Jul. 20, 2012.

(51) Int. Cl. A61B 17/84

(52) U.S. Cl. CPC A61B 17/846
USPC 606/319; 606/329

(57) ABSTRACT

The present disclosure provides methods and devices that allow for rotational control, compression and fusion of the ankle as required for treatment while providing additional mobility in the movement of the foot. The devices of the present disclosure are configured to go through the talus and tibia for fusion of ankle joint while avoiding the calcaneus and subtalar joint. According to a first aspect, exemplary embodiments of the present disclosure are curved to provide sufficient area for rotational control and compression of the ankle bones. According to a second aspect, exemplary embodiments of the nails of the present disclosure are configured to be placed at an angle with respect to the intramedullary canal.

### CROSS-REFERENCE TO RELATED APPLICATIONS

The present application claims priority to co-pending U.S. Provisional Patent Application No. 61/673,949, entitled “ANKLE NAIL ASSEMBLY,” filed Jul. 20, 2012, the disclosure of which is hereby incorporated herein by reference in its entirety.

### TECHNICAL FIELD

The present disclosure relates generally to methods and medical devices to fuse bones that form a joint, and more particularly relates to methods and medical devices for fusing bones in an ankle joint.

### BACKGROUND

In certain circumstances, a joint, and more particularly an ankle joint, is so degraded that it becomes necessary to prevent the joint from moving because other, less-severe surgical operations, such as those that include implanting an ankle prosthesis, would be ineffective. The procedure to fuse bones to immobilize the joint is called arthrodesis.

The procedure effectively eliminates almost all mobility of a joint. While there are many methods currently available to hold the bones together until they grow into each other (fuse) to become one bone, they are not without disadvantages. One method places cannulated screws across the ankle, and the compression of the screws causes the ankle to fuse. This method requires precise positioning of the screws so they do not intersect one another while avoiding improper placement that can result in ankle misalignment.

A second method is fusion with a plate that is secured to the ankle until fusion occurs. While the placement of the plate during surgery is easier, the size of the wound presents an issue because installation of the plate requires larger operating areas on the foot. The third method includes a fusion nail that is inserted through a smaller opening on the bottom of the foot into the intramedullary canal of the tibia, which impacts the movement of the subtalar joint and can result in the patient having a club foot gate.

### BRIEF SUMMARY

The present application provides methods and medical devices for fusing bones in an ankle joint. In accordance with one embodiment, a bone fusion nail is provided. The bone fusion nail has a first end, a second end, and an arcing elongated structure extending therebetween, configured such that, when inserted, the fusion nail extends through the talus and tibia of a subject and fuses the ankle joint between the talus and tibia. Another embodiment provides for a bone fusion nail having a first end a second end, with a straight elongated structure extending therebetween, configured such that, when inserted, the fusion nail extends through the talus and tibia of a subject and fuses the ankle joint between the talus and tibia.

In accordance with one embodiment, a method for fusing an ankle joint is provided. The method includes drilling an arcical hole between a talus and a tibia of a subject and inserting an arcical fusion nail which is configured to extend between the talus and tibia of the subject and to fuse an ankle joint therebetween. Another embodiment includes drilling a transaxial hole between a talus and a tibia of a subject and inserting an transaxial fusion nail which is configured to extend between the talus and tibia of the subject and to fuse an ankle joint therebetween.

The foregoing has outlined rather broadly the features and technical advantages of the present invention in order that the detailed description that follows may be better understood. Additional features and advantages will be described hereinafter which form the subject of the claims. It should be appreciated by those skilled in the art that the conception and specific embodiment disclosed may be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of embodiments described herein, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present embodiments.

### BRIEF DESCRIPTION OF THE DRAWINGS

For a more complete understanding of the disclosed methods and apparatuses, reference should be made to the embodiments illustrated in greater detail in the accompanying drawings, wherein:

FIG. 1 is a lateral view of a left foot, ankle and lower leg skeleton;

FIG. 2 is a medial view of a left foot, ankle and lower leg skeleton;

FIG. 3 is a back, or posterior, view of a left foot, ankle and lower leg skeleton;

FIGS. 4A-4C are a side view of an embodiment of an exemplary fusion nail according to the first aspects of the present disclosure;

FIGS. 5A and 5B are a back, or posterior, view of a left foot, ankle and lower leg skeleton with the fusion nail of FIG. 4 positioned according to the aspects of the present disclosure;

FIG. 5C is a back, or posterior, view of a left foot, ankle and lower leg skeleton with two fusion nails of FIG. 4 positioned according to the aspects of the present disclosure;

FIG. 6 is a side view an exemplary fusion nail coupled to a distal jig according to the first aspects of the present disclosure;

FIGS. 7A and 7B are a back, or posterior, view of a left foot, ankle and lower leg skeleton with a fusion nail according to the second aspects of the present disclosure positioned therein;

FIGS. 7C-7E are a side view of an embodiment of an exemplary fusion nail according to the second aspects of the present disclosure; and

FIG. 8 is a perspective view an exemplary fusion nail coupled to a distal jig according to the first aspects of the present disclosure.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DETAILED DESCRIPTION

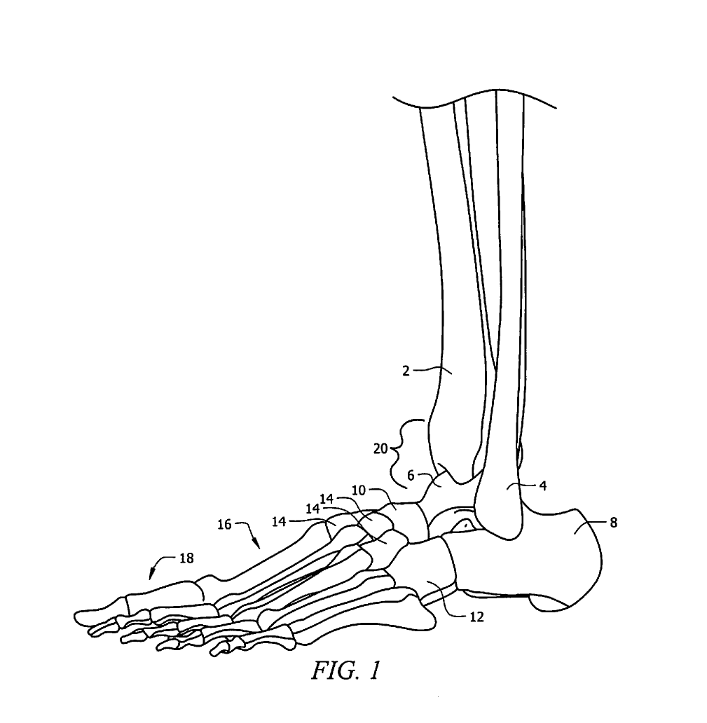

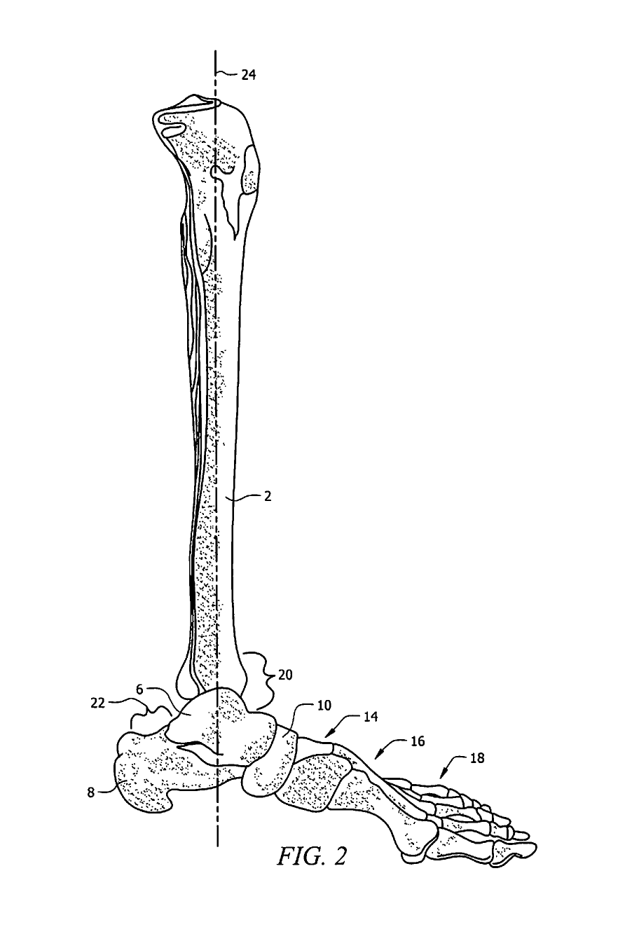

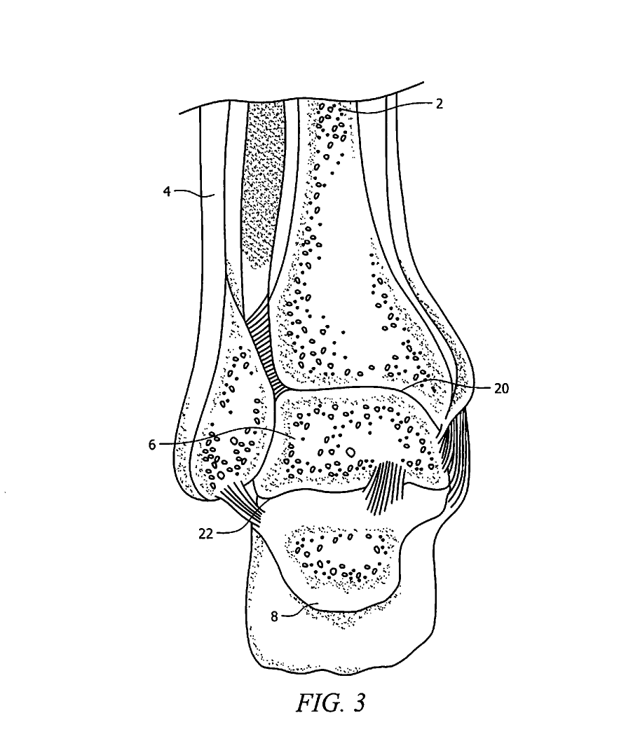

FIG. 1 illustrates a lateral view (outside view) of the skeleton of a left foot, ankle and distal leg portion. FIG. 2 illustrates a medial lateral view (inside view) of a left foot, ankle and lower leg skeleton. FIG. 3 illustrates a back, or posterior, view of the skeleton of a left foot, ankle and distal leg portion. Referring to FIGS. 1 and 2, the distal leg portion includes tibia 2 and fibula 4. The bones of the ankle and foot include: talus 6, calcaneus 8, navicular 10, cuboid 12, cuneiforms 14, metatarsals 16, and phalanges 18. In particular, ankle joint 20 comprises tibia 2, fibula 4, and talus 6. Referring to FIGS. 1 and 2, tibia 2 forms the inside, or medial, portion of ankle joint 20, fibula 4 forms the lateral, or outside portion of ankle joint 20 and talus 6 is the bone located underneath tibia 2 and fibula 4. In the preferred embodiment, the proffered procedures typically immobilize joint 20 by fusing tibia 2 and talus 6.

Referring to FIGS. 1 and 2, subtalar joint 22 is the meeting point between talus 6 and calcaneus 8. Referring to FIG. 1, prior art methods involving fusion nails typically drive the fusion nail from the bottom of the foot through subtalar joint 22 and into the intramedullary canal of tibia 2, as indicated by line 24. The placement of the fusion nail through subtalar joint 22 of prior art methods limits movements in the foot. The present disclosure provides methods and devices, particularly fusion nails, that allow for compression and fusing of the ankle as required while providing additional mobility in the foot with fusion nails that avoid subtalar joint 22 when placed in a patient. It is noted that the term “nail” is used for convenience (and in some instances may be used interchangeably with the term “fastener”), and as described in the present application the nail may comprise threaded portions and the like. The fusion nails of the present disclosure are configured to go through talus 6 and tibia 2 for fusion of ankle joint 20 while avoiding calcaneus 8 and subtalar joint 22. According to a first aspect, exemplary embodiments of the fusion nails of the present disclosure are curved to provide compression of the ankle bones. According to a second aspect, exemplary embodiments of the nails of the present disclosure are configured to be placed at an angle with respect to the intramedullary canal.

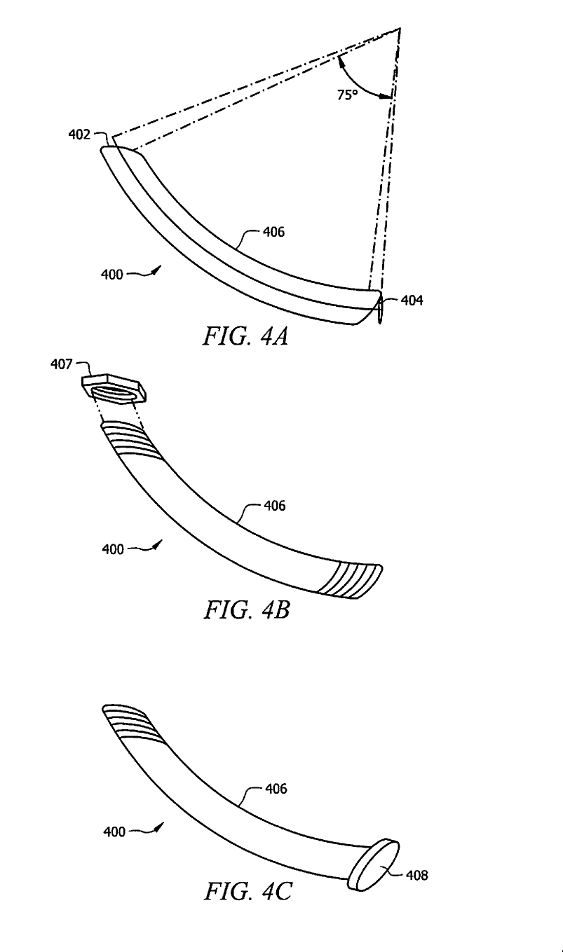

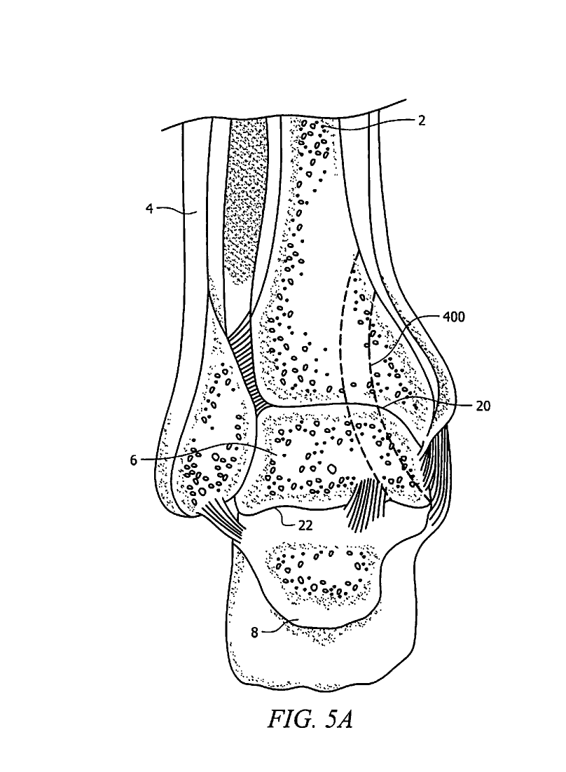

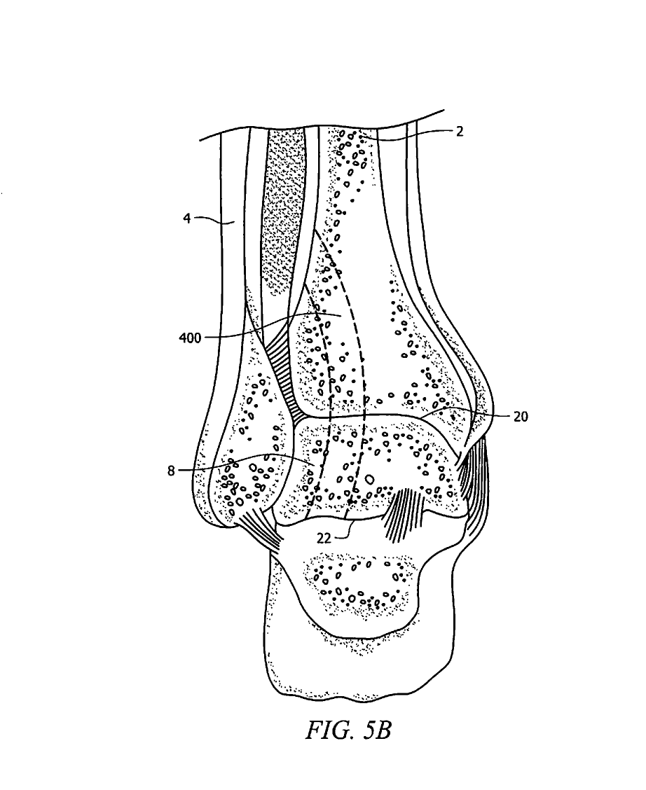

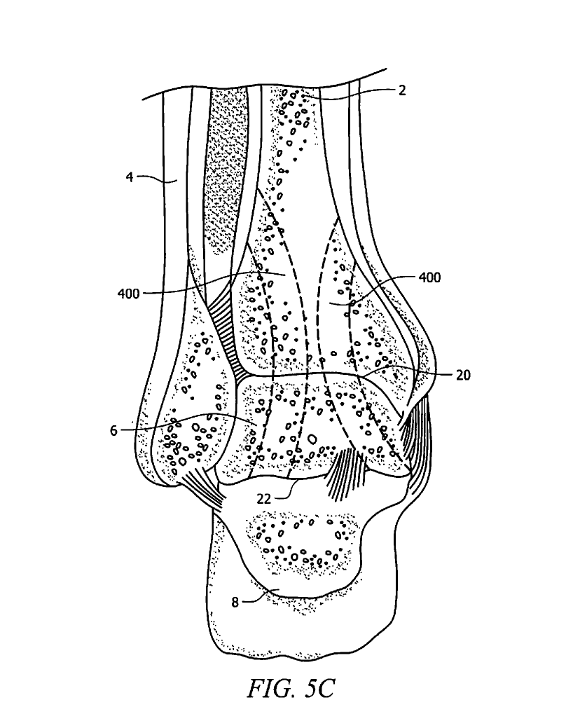

FIGS. 4A-4C show exemplary fusion nails, fusion nail 400, in accordance with aspects of the present disclosure. FIGS. 5A and 5B show the posterior view of ankle joint 20 with fusion nail 400 positioned in ankle joint 20. FIGS. 5A and 5B show fusion nail 400 extended across tibia 2 and talus 6 to effect fusion of ankle joint 20 while avoiding calcaneus 8 and subtalar joint 22. Fusion nail 400 is an elongated structure having first end 402, second end 404, and body 406 extending there between. Depending on the point of insertion, first end 402 may be the distal end and second end 404 may be the proximal end, or vice versa. In the preferred embodiment, fusion nail 400 has an arc with an angle of about 75 degrees. Body 406 of fusion nail 400 is preferably generally cylindrical and has a diameter of preferably about 7 mm-12 mm. In one embodiment, fusion nail 400 has an arc length of about 8 cm-10 cm.

Referring to FIG. 5A, in one embodiment, fusion nail 400 is positioned toward the medial side of tibia 2 and talus 6. Preferably, fusion nail 400 is inserted from the medial distal portion of talus 6, extends through talus 6 and tibia 2 and curve back to exit the medial side of tibia 2, proximal to the point of entry. Alternatively, fusion nail 400 is inserted from the medial side of tibia 2, extends through tibia 2 and talus 6 and curve back to exit through the medial distal portion of talus 6, distal to the point of entry.

Referring to FIG. 5B, in another embodiment, fusion nail 400 is positioned toward the lateral side of tibia 2 and talus 6. Preferably, fusion nail 400 is inserted from the lateral distal portion of talus 6, extends through talus 6 and tibia 2 and curve back to exit the lateral side of tibia 2, proximal to the point of entry. Alternatively, fusion nail 400 is inserted on the lateral side of tibia 2, extends through tibia 2 and talus 6 and curve back to exit through the lateral distal portion of talus 6, distal to the point of entry.

Referring to FIG. 5C, certain embodiments can include two fusion nails 400 if additional compression is desired or required for bone fusion. One fusion nail 400 is positioned near the lateral side of tibia 2 and talus 6, and the other fusion nail 400 is positioned near the medial side of tibia 2 and talus 6. Fusion nails 400 in FIG. 5C can be inserted as described for fusion nails 400 in FIGS. 5A and 5B. The descriptions of the insertion and position corresponding to FIGS. 5A and 5B are equally applicable to FIG. 5C, and thus are not repeated. Referring to FIGS. 5A, 5B, and 5C, fusion nail 400 is generally curved so that, when inserted, first and second ends 402 and 404 are oriented toward the same side.

As shown by the embodiments in FIGS. 5A, 5B, and 5C, the generally curved shape of fusion nail 400 allows it to penetrate ankle joint 20 sufficiently to compress ankle joint 20 and hold tibia 2 and talus 6 in place for fusion while providing a large contact area with tibia 2 and talus 6 to counteract shear forces exerted by tibia 2 and talus 6. In the preferred embodiment, a single fusion nail 400 is sufficient to effect bone fusion between tibia 2 and talus 6. The generally curved shape of fusion nail 400 further provides the benefit of ease of removal of fusion nail 400 if it breaks in half. Removal can be done by pulling one half out of the end through tibia 2 and the other out of the end through talus 6. With prior art nails that are inserted into the intramedullary canal of tibia 2, this is not possible, and more complex removal surgery is needed.

In one embodiment, to allow a user to impose additional compression force on ankle joint 20, at least one of first end 402 and second end 404 can further include external threads (see FIGS. 4B and 4C), which are complementary to internal threads on a compression nut member 407. The threaded end of fusion nail 400 preferably extends beyond the surface of the respective bone to allow the compression nut member to engage with fusion nail 400. When additional compression force is desired, the user engages the compression nut member with the threaded end of fusion nail 400 and tightens the compression nut member until the desired compression is achieved. The tightening is preferably done with an appropriate tool, such as a wrench of complementary shape with the compression nut member. Tightening of the compression nut member pulls tibia 2 and talus 6 closer to one another and hold them together in that configuration until fusion is achieved.

If only one end of fusion nail 400 is threaded, the other end is preferably capped with a head 408 that has a larger diameter than body 406 to provide compression and prevent the tightening of the threaded end from pulling of fusion nail 400 toward the threaded end. If both ends of fusion nail 400 are threaded, two compression nut members can be provided and tightened until the desired compression is achieved.

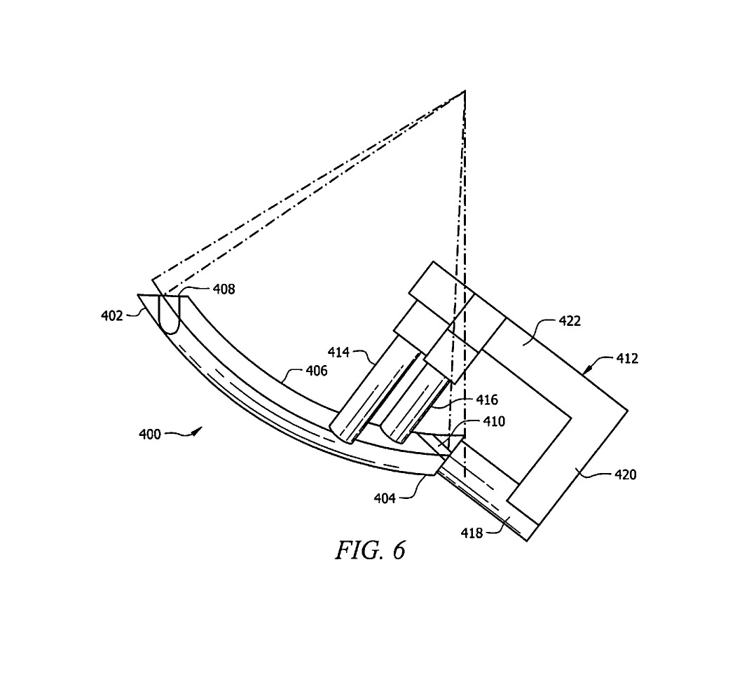

Referring to FIG. 6, in another embodiment, compression screws provide additional compression instead of threaded end(s) and compression nut members. Instead of having external threads, at least one of first end 402 and second end 404 further includes internal threads 408 and 410 at first end 402 and second end 404, respectively. Internal threads 408 preferably extend across body 406 to allow a compression screw to be inserted and tightened while the head of the compression screw sits tight against the surface of the respective bone, e.g., tibia 2. Internal threads 410 preferably extend into body 406 also to allow a compression screw to be inserted and tightened while the head of the compression screw sits tight against the surface of the respective bone, e.g., talus 6. Tightening of the compression screws forces tibia 2 and talus 6 closer together and hold them tightly in that configuration until fusion is achieved.

Alternatively, internal threads 408 and 410 can run substantially along the axis of body 406 provided that the head of the compression screw is configured to sit tight against the surface of the respective bone. For instance, the head of the compression screw may be angled with respect to the body of the screw to match the angle of the medial side of tibia 2 instead of the standard substantially perpendicular configuration. Further, in one embodiment, internal threads 408 and 410 can also serve as the coupling point for the respective jigs or position guidance components used to insert fusion nail 400 or other elements as further described below.

Fusion nail 400 may experience rotational force during use, which would detract from the compression of ankle joint 20 and potentially loosen any compression elements (e.g., compression nuts or screws) if provided, as well as expose fusion nail 400 to greater risk of breakage. To reduce or prevent the rotational force exerted on fusion nail 400, one or more locking screws (not shown) can be coupled to fusion nail 400 to provide additional rotation control.

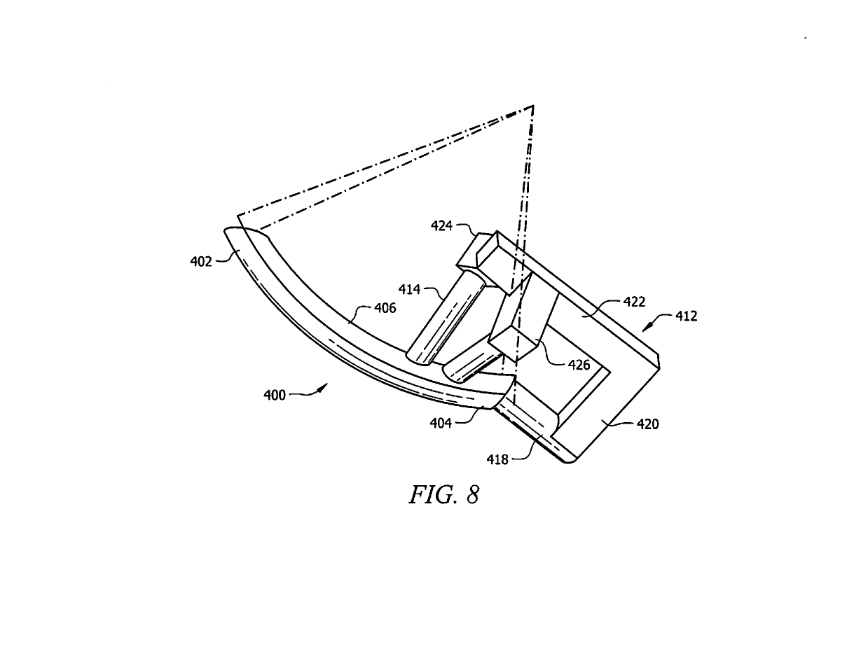

In the preferred embodiment, fusion nail 400 further includes two distal locking screws near the distal end of fusion nail 400. FIGS. 6 and 8 show fusion nail 400 coupled to distal jig 412, which is used to insert the distal locking screws, as further discussed below. Channels 414 and 416 indicate the preferred position and location of the distal locking screws on body 406 of fusion nail 400. The interlocking screws are preferably positioned so that they form about a 30 degrees skewed angle between one another. One locking screw preferably extends toward the antero-medial portion of the respective bone, e.g., talus 6, while the other locking screw preferably extends toward the postero-medial portion of the respective bone, e.g., talus 6. In one embodiment, one or both locking screws can traverse through both tibia 2 and talus 6. The end of one or both locking screws can extend through body 406 or disposed in body 406. Body 406 preferably provides a complementary channel through body 406 at the proper angle to receive and hold the external threads of the end of the locking crews as they engage the patient's bone and body 406 when tightened. In another embodiment, fusion nail 400 includes one or more proximal locking screws near the proximal end of fusion nail 400 instead of, or in addition to, the distal locking screws. The descriptions of the distal locking screws are equally applicable to the proximal locking screws and thus are not repeated. It is understood that the number of locking screw can vary to provide the appropriate or desired rotation control.

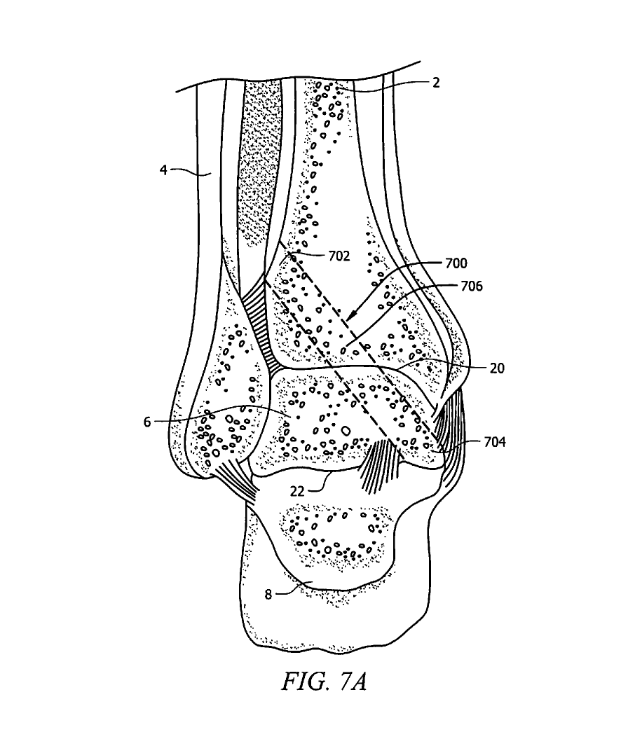

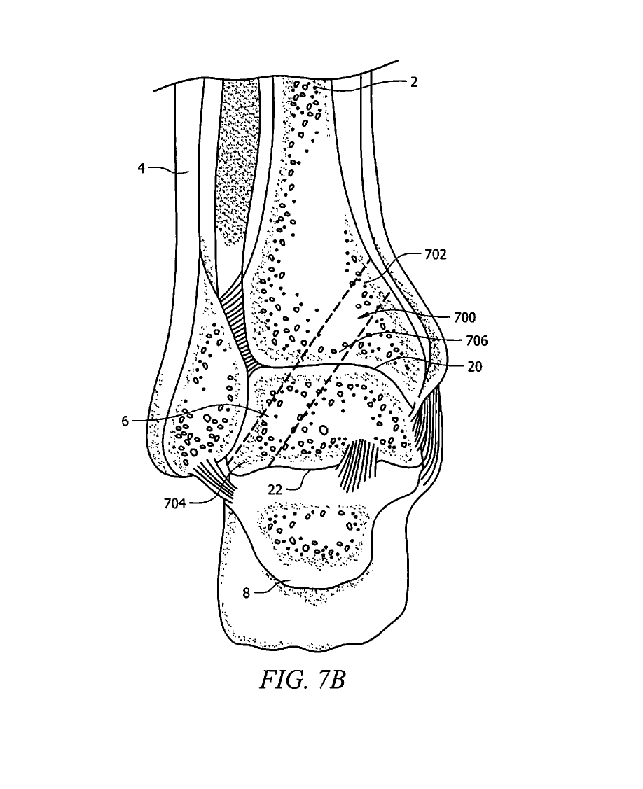

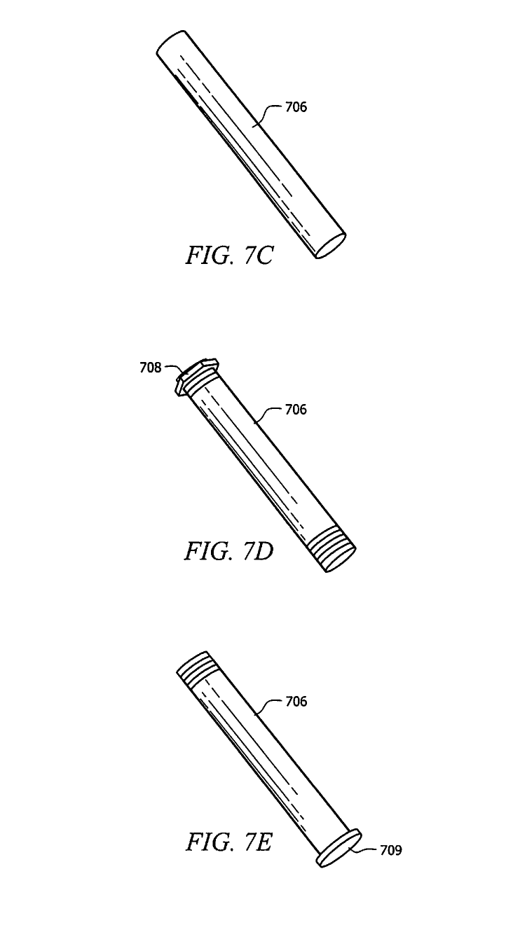

According to a second aspect of the present disclosure, there is provided a fusion nail that is inserted at an angle. In some embodiments, the insertion may be made so as to avoid the subtalar joint. Referring to FIGS. 7A and 7B, fusion nail 700 extends across tibia 2 and talus 6 to effect fusion of ankle joint 20. In the illustrated embodiment, nail 700 avoids calcaneus 8 and subtalar joint 22. Fusion nail 700 is an elongated structure having proximal end 702, distal end 704, and body 706 extending therebetween. In the preferred embodiment, fusion nail 700 is generally straight. Body 706 of fusion nail 700 is preferably cylindrical and has a diameter of preferably about 7 mm-12 mm. In one embodiment, fusion nail 700 has a length of about 8 cm-10 cm. FIGS. 7C-7E illustrate various embodiments of fusion nail 700 which may be inserted as shown in FIGS. 7A and 7B, it is noted that while these figures show that the ends of the fusion nail are straight, such ends may be angled (such as shown in by the dotted portion of FIGS. 7A and 7B) so as to reduce the distance that the fusion nail extends from its respective bone portion at its ends.

Referring to FIG. 7A, in one embodiment, fusion nail 700 is positioned diagonally, extending from the lateral side of tibia 2 to the medial proximal portion of talus 6. Preferably, fusion nail 700 is inserted from the medial distal portion of talus 6, extends through talus 6 and tibia 2 and exits through the lateral side of tibia 2. Alternatively, fusion nail 700 is inserted from the lateral side of tibia 2, extends through tibia 2 and talus 6 and exits through the medial distal portion of talus 6.

Referring to FIG. 7B, in another embodiment, fusion nail 700 is positioned diagonally, extending from the medial side of tibia 2 to the lateral distal portion of talus 6. Preferably, fusion nail 700 is inserted at the medial side of tibia 2, extends through talus 6 and tibia 2 to exit near the lateral distal side of talus 6. Alternatively, fusion nail 700 is inserted near the lateral proximal portion of talus 6, extends through tibia 2 and talus 6, and exits through the medial side of tibia 2.

As shown by the embodiments in FIGS. 7A and 7B, the diagonal positions of fusion nail 700 allow it to penetrate ankle joint 20 to compress and hold the joint in place for fusion while providing a large contact area with tibia 2 and talus 6 to counteract shear forces exerted by tibia 2 and talus 6 of ankle joint 20. In the preferred embodiment, a single fusion nail 700 is sufficient to effect bone fusion between tibia 2 and talus 6. Because proximal end 702 extends through the surface of tibia 2 and distal end 704 extends through the surface of talus 6, this configuration provides the benefit of ease of removal of fusion nail 400 if it breaks in half or when sufficient fusion is achieved. If fusion nail 700 breaks, removal can be done by pulling one half out of the end through tibia 2 and the other out of the end through talus 6. With prior art nails that are inserted into the intramedullary canal of tibia 2, this is not possible, and more complex removal surgery is needed.

In one embodiment, to allow a user to impose additional compression force on ankle joint 20, at least one of proximal end 702 and distal end 704 can further include external threads (FIG. 7D), which are complementary to internal threads on a compression nut member 708. The threaded end of fusion nail 700 preferably extends beyond the surface of the respective bone to allow the compression nut member 708 to engage with fusion nail 700. When additional compression force is desired, the user engages the compression nut member with the threaded end of fusion nail 700 and tightens it until the desired compression is achieved. The tightening is preferably done with the appropriate tool, such as a wrench. Tightening of the compression nut member pulls tibia 2 and talus 6 closer to one another and hold them together in that configuration until fusion is achieved.

If only one end of fusion nail 700 is threaded, the other end is preferably capped with a head 709 that has a larger diameter than body 706 to provide compression and prevent the tightening of the threaded end from pulling of fusion nail 700 toward the threaded end. If both ends of fusion nail 700 are threaded, two compression nut members can be provided and tightened until the desired compression is achieved.

In another embodiment, compression screws provide additional compression instead of threaded end(s) and compression nut members. Instead of having external threads, at least one of proximal end 702 and distal end 704 further includes internal threads similar to internal threads 408 and 410 of fusion nail 400 described with respect to FIG. 6. Tightening of the compression screws forces tibia 2 and talus 6 closer together and hold them tightly in that configuration until fusion is achieved.

Alternatively, the internal threads of fusion nail 700 can run substantially along the axis of body 706 provided that the head of the compression screw is configured to sit tight against the surface of the respective bone. For instance, the head of the compression screw may be angled with respect to the body of the screw to match the angle of the medial side of tibia 2 instead of the standard perpendicular configuration. Further, in one embodiment, the internal threads of fusion nail 700 can also serve as the coupling point for the respective jigs or position guidance system components used in inserting fusion nail 700 or other elements as further described below.

Fusion nail 700 may experience rotational force during use, which would detract from the compression of ankle joint 20 and potentially loosen any compression elements (e.g., compression nuts or screws) if provided, as well as expose fusion nail 700 to greater risk of breakage. To reduce or prevent the rotational force exerted on fusion nail 700, one or more locking screws (not shown) can be coupled to fusion nail 700 to provide additional rotation control, as described above for fusion nail 400. The descriptions relating to the distal and proximal locking screws of fusion nail 400 are equally applicable provide additional rotation control of fusion nail 700, and thus are not repeated.

Insertion methods known to those skilled in the art can be used to place fusion nails 400 and 700 into a patient's foot. In one exemplary embodiment, a guidance system configured to the dimensions and desired placement of nail 300 is used. Typical guidance systems includes components known as jigs. An insertion jig (not shown) is used to drill a hole of appropriate dimensions at the appropriate location for the placement of fusion nail 400 or 700. The hole is preferably of the same dimensions as the nail being inserted. The insertion jig is configured to be positioned about tibia 2 and talus 6. The insertion jig preferably has at least three arms extending therefrom to provide positioning guidance for drilling of the hole that will house fusion nail 400 or 700. The first arm contacts the patient's leg at the desired exit point. The second arm contacts the patient's leg at the point of entry. Accordingly, the first and second arms allow the user to dictate where to place fusion nail 400 or 700 in tibia 2 and talus 6 vertically, e.g., proximally toward tibia 2 or distally toward talus 6, and horizontally, e.g., toward the anterior or posterior. However, this still leaves a broad range of entry and exit angles where body 406 or 706 can point toward the anterior, posterior or laterally. The third arm of the insertion jig is configured to fix the entry and exit points at the proper angles. In the preferred embodiment, the third arm is at an angle to the first and second arms so that when the third arm contacts the patient's leg, the angle of the first and second arms with respect to the applicable anterior or posterior plan is defined. This allows the care provider or user to place the hole, and thus, fusion nail 400 or 700, at the precise location before the hole is drilled where prior art methods typically involve taking X-rays around a patient's ankle as the hole is being drilled to verify the proper placement.

Coupled to the second arm at the point of entry is a hollow shaft that has substantially the same shape and dimensions as the fusion nail being inserted. The hollow shaft is provided at the angle A reamer or drill bit of substantially the same shape and dimensions as the respective fusion nail is inserted through the hollow shaft, which guides the reamer or drill bit to enter the bone at the proper angle as the reamer goes through tibia 2 and talus 6 and exits to the first arm.

Fusion nail 400 or 700 should fit snugly in the hole provided by the reamer. The inserted fusion nail preferably includes internal threads disposed in the body of the fusion nail at both ends to allow coupling of additional jigs, such as the compression jig to add additional compression force if desired or distal and proximal jigs to insert locking screws.

If additional compression is needed or desired, the compression jig is used where the compression jig is configured to hold the nail at one end so that threaded compression elements (e.g., compression nut members or screws) can be tightened at the other end. For instance, the compression jig can be complementary to the head of the nail, if one is provided.

If additional rotational control is needed, the distal jig and/or proximal jig can be used to insert the locking screws. Like the insertion jig, the distal jig and proximal jig provide the user (e.g., care provider) with provider with simple and user-friendly placement aid. The distal jig and proximal jig provide the precise location and angle to drill the hole for the respective locking screws. Referring to FIGS. 6 and 8, for an embodiment with at least two locking screws, distal jig 412 can be used. As shown, distal jig 412 couples to distal end 404. Distal jig 412 has three arms: 418, 420, and 422. Arm 418 extends vertically away from distal end 404 of fusion nail 400. The length of each arm is configured to place locking screw arms 424 and 426 at the precise location relative to the respective holes on body 406 receiving the locking screws. Locking screw arms 424 and 426 extend from arm 422 at the desired angle at which the locking screws are to be inserted. In a preferred embodiment, channels 414 and 416 allow the locking screws enter the respective bone and engage body 406 at the desired angles. That is, the locking screws are inserted into the corresponding channel 414 or 416 and are tightened until the desired or required rotational control is achieved. Channels 414 and 416 are preferably formed by drilling through the respective bone at the desired angles. In the preferred embodiment, locking screw arms 424 and 426 allow channels 414 and 416 to be formed at the desired angles. Sliding sleeves or hollow shafts (not shown) engage locking screw arms 424 and 426 at a designated location. The sliding sleeves or hollow shafts preferably have substantially the same shape and dimensions as the respective locking screws. In one embodiment, a reamer or drill bit of substantially the same shape and dimensions as the respective locking screw is inserted through a hollow shaft, which guides the reamer or drill bit to enter the bone at the proper angle as the reamer goes through the respective bone to form channels 414 and 416. In another embodiment, a nested cannulated guide is used to form channels 414 and 416. The nested cannulated guide preferably has a series of sliding sleeves disposed or nested inside each other. The smallest sleeve has an inner diameter configured for a drill bit to initiate the process by providing a small channel through the bone. The biggest sleeve has an inner diameter that essentially matches the diameter of the screw head of the locking screws. This allows for drilling to form channels 414 and 416 with the properly sized diameter to allow locking screws to be inserted. The locking screws should be able to be inserted into channels 414 and 416 formed as described above with the use of distal jig 412 to engage body 406 and provide rotation control. If proximal locking screws are needed in addition, or alternatively, to distal locking screws, a proximal jig that functions the same way as distal jig 412 to provide the precise placement of the proximal locking screws can be used. The descriptions of distal jig 412 are equally applicable to the proximal jig, and thus are not repeated.

If additional compression is needed the distal or proximal jig can be used to place the appropriate interlock screws and the opposite compression screw or nut can be tightened against the interlock screws. This is particularly helpful for poor bone quality.

Although the embodiments of the present disclosure and their advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. An apparatus comprising:
a bone fusion nail having a first end, a second end, and an arcing elongated structure extending therebetween, said bone fusion nail configured such that, when inserted, the fusion nail extends through the talus and tibia of a subject and fuses the ankle joint between the talus and tibia.

2. The apparatus of claim 1 wherein the first end is configured to be inserted into a medial surface of the tibia and extend through the talus and curve back toward a medial surface of the talus.

3. The apparatus of claim 1 wherein the first end is configured to be inserted into a medial surface of the talus and extend through the tibia and curve back toward a medial surface of the tibia.

4. The apparatus of claim 1 wherein the first end is configured to be inserted into a lateral surface of the tibia and extend through the talus and curve back toward a lateral surface of the talus.

5. The apparatus of claim 1 wherein the first end is configured to be inserted into a lateral surface of the talus and extend through the tibia and curve back toward a lateral surface of the tibia.

6. The apparatus of claim 1 wherein the arcing elongated structure has an arc angle of about 75 degrees.

7. The apparatus of claim 1 wherein the elongated structure has an arc length of about 8-10 centimeters.

8. The apparatus of claim 1 wherein the elongated structure has a diameter of about 7-12 centimeters.

9. The apparatus of claim 1 wherein at least one of the first end and second end comprises a threaded portion configured to extend beyond the surface of a respective bone when inserted and to engage with a nut member.

10. The apparatus of claim 9 wherein at least one of the first and second end comprises a capped end having a diameter larger than the diameter of the elongated structure.

11. The apparatus of claim 1 wherein the bone fusion nail is configured to engage at least one locking screw.

12. The apparatus of claim 1 further comprising at least one jig configured to guide the insertion of at least one of the fusion nail and a locking screw.

13. A method for fusing an ankle joint, the method comprising:
drilling an arcical hole between a talus and a tibia of a subject; and
inserting an arcical fusion nail which is configured to extend between the talus and tibia of the subject and to fuse an ankle joint therebetween.

14. The method of claim 13 wherein inserting the arcical fusion nail comprises inserting the nail into a medial surface of the tibia and extending the nail through the talus and to curve back toward a medial surface of the talus.

15. The method of claim 13 wherein inserting the arcical fusion nail comprises inserting the nail into a medial surface of the talus and extending the nail through the tibia and to curve back toward a medial surface of the tibia.

16. The method of claim 13 wherein inserting the arcical fusion nail comprises inserting the nail into a lateral surface of the tibia and extending the nail through the talus and to curve back toward a lateral surface of the talus.

17. The method of claim 13 wherein inserting the arcical fusion nail comprises inserting the nail into a lateral surface of the talus and extending the nail through the tibia and to curve back toward a lateral surface of the tibia.

18. The method of claim 13 further comprising inserting at least on locking screw, the locking screw configured to engage the arcical fusion nail.

19. The method of claim 13 further comprising:
drilling a plurality of arcical hole between a talus and a tibia of a subject; and
inserting a plurality of arcical fusion nails which are configured to extend between the talus and tibia of the subject and to fuse an ankle joint therebetween.

20. The method of claim 13 wherein at least one end of the fusion nail comprises a threaded portion configured to extend beyond the surface of a respective bone when inserted and to engage with a nut member.

21. The method of claim 20 wherein at least one end of the fusion nail comprises a capped end having a diameter larger than the diameter of the fusion nail.

22. An apparatus comprising:
a bone fusion nail having a first end, a second end, and an elongated structure extending therebetween, said bone fusion nail configured such that, when inserted, the fusion nail extends through the talus and tibia of a subject and fuses the ankle joint between the talus and tibia.

23. The apparatus of claim 22 wherein the first end is configured to be inserted into a laterial surface of the tibia and extend through the talus.

24. The apparatus of claim 22 wherein the first end is configured to be inserted into a laterial surface of the talus and extend through the tibia.

25. The apparatus of claim 22 wherein the first end is configured to be inserted into a medial surface of the tibia and extend through the talus.

26. The apparatus of claim 22 wherein the first end is configured to be inserted into a medial surface of the talus and extend through the tibia.

27. The apparatus of claim 22 wherein at least one of the first end and second end comprises a threaded portion configured to extend beyond the surface of a respective bone when inserted and to engage with a bolt or nut member.

28. The apparatus of claim 27 wherein at least one of the first and second end comprises a capped end having a diameter larger than the diameter of the elongated structure.

29. The apparatus of claim 22 wherein the bone fusion nail is configured to engage at least one locking screw.

30. The apparatus of claim 22 wherein the elongated structure is an arcical structure.

31. A method for fusing an ankle joint, the method comprising:
drilling a transaxial hole between a talus and a tibia of a subject; and
inserting an transaxial fusion nail which is configured to extend between the talus and tibia of the subject and to fuse an ankle joint therebetween.

32. The method of claim 31 wherein inserting the transaxial fusion nail comprises inserting the nail into a lateial surface of the tibia and extending the nail through the talus.

33. The method of claim 31 wherein inserting the transaxial fusion nail comprises inserting the nail into a laterial surface of the talus and extending the nail through the tibia.

34. The method of claim 31 wherein inserting the transaxial fusion nail comprises inserting the nail into a medial surface of the tibia and extending the nail through the talus.

35. The method of claim 31 wherein inserting the transaxial fusion nail comprises inserting the nail into a medial surface of the talus and extending the nail through the tibia.

36. The method of claim 31 further comprising inserting at least on locking screw, the locking screw configured to engage the transaxial fusion nail.

37. The method of claim 31 further comprising:
drilling a plurality of transaxial hole between a talus and a tibia of a subject; and
inserting a plurality of transaxial fusion nails which are configured to extend between the talus and tibia of the subject and to fuse an ankle joint therebetween.
